(19) United States
(12) Patent Application Publication
(10) Pub. No.: US 2024/0395373 A1
(43) Pub. Date: Nov. 28, 2024

(54) CONCEPT MAPPING FOR INTEGRATED CHARTING

(71) Applicant: Cerner Innovation, Inc., Kansas City, MO (US)

(72) Inventors: Suman Pal, Bangalore (IN); Monica Gaur, Delhi (IN); Rupanjali Chaudhuri, Bangalore (IN); Vadim Khotilovich, Leawood, KS (US); Pragnya Ranjan Pradhan, Similipada (IN); Oshin Benny Anto, Kerala (IN); Kalaivanan R, Bangalore (IN); Chetan KV, Bangalore (IN)

(73) Assignee: Cerner Innovation, Inc., Kansas City, MO (US)

(21) Appl. No.: 18/321,938

(22) Filed: May 23, 2023

(51) Int. Cl. G16H 10/60; G06F 40/284

(52) U.S. Cl. CPC G16H 10/60; G06F 40/284

(57) ABSTRACT

Techniques for presenting recommendations of candidate unmapped proprietary codes for target standard codes are disclosed. The techniques include comparing datasets of unmapped proprietary codes with datasets of a target standard code. The datasets are represented as vector embeddings generated using word embedding techniques. Cosine similarities between the vector embeddings of candidate proprietary codes and the target standard code are used to identify and rank a list of candidate unmapped proprietary codes for the target standard code. The cosine similarity scores may be weighted.

### TECHNICAL FIELD

The present disclosure relates to ontological mapping for integrated charting. In particular, the present disclosure relates to methods and systems of ontological mapping using natural language processing.

### BACKGROUND

With the increasing adoption of Electronic Health Records (EHRs), healthcare Information Technology (IT) is under a deluge of increasing velocity, volume, and variety of data. The challenge is that data lives across multiple systems, applications, databases, and ontologies. This data needs to be brought together to build consistent knowledge bases for rendering value through interoperability. Different concepts, terminologies, and data models need to be reconciled as part of this process. When a product has higher semantic expressivity, it becomes more interoperable. In other words, interoperability enables the use of health information systems across organizational boundaries, and it also enables seamless integration in the workflow.

Interoperability can be defined as the ability of different information systems, devices and applications (systems) to access, exchange, integrate and cooperatively use data in a coordinated manner, within and across organizational, regional and national boundaries, to provide timely and seamless portability of information and optimize the health of individuals and populations globally.

The primary objective of any healthcare IT organization is the curation of interoperable data and intelligence across the healthcare that consumers can trust, leverage, enrich and integrate. For this to happen, Semantic Interoperability (SI) is indispensable. SI is the ability to use digital health records across diverse care settings and clinical software that use different words to say the same thing. Systems in the hospitals should be able to communicate with one another at the data level. To enable seamless communication, standardization of clinical events across different clinical software systems and in diverse care settings is necessary as the same condition or diagnosis is often described using different words, aka synonyms.

These challenges are faced outside of healthcare sectors, including in e-commerce and in food and beverage.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIG. 2 illustrates an example set of operations for presenting recommendations of candidate unmapped proprietary codes in accordance with one or more embodiments;

FIG. 3 illustrates an example of data flow during an example set of operations for presenting a recommendation of candidate unmapped proprietary codes;

FIG. 4 illustrates an interface for presenting recommendations of candidate standardized event codes; and

FIG. 5 shows a block diagram that illustrates a computer system in accordance with one or more embodiments.

### DETAILED DESCRIPTION

In the following description, for the purposes of explanation, numerous specific details are set forth in order to provide a thorough understanding. One or more embodiments may be practiced without these specific details. Features described in one embodiment may be combined with features described in a different embodiment. In some examples, well-known structures and devices are described with reference to a block diagram form in order to avoid unnecessarily obscuring the present invention.
1. GENERAL OVERVIEW
2. EVENT CODE MAPPING SYSTEM
3. PRESENTING RECOMMENDATIONS OF CANDIDATE UNMAPPED PROPRIETARY CODES FOR MAPPING TO STANDARD CODES
4. EXAMPLE EMBODIMENT
5. RECOMMENDATION INTERFACE
6. HARDWARE OVERVIEW
7. MISCELLANEOUS; EXTENSIONS

### 1. General Overview

One or more embodiments generate recommendations of candidate unmapped proprietary codes for storing in association with a standard code. The unmapped proprietary codes, as referred to herein, include codes used by specific organizations or vendors that have not been mapped to a standard code. The standard codes, as referred to herein, include codes in which each standard code is directed to a single concept-concepts that might not otherwise have an industry standardized code. The standard codes, as referred to herein, are mapped to one or more mapped proprietary codes.

Initially, the system generates vector embeddings for the unmapped proprietary codes by applying a vector embedding function to the unmapped propriety codes. Applying a vector embedding function to the unmapped proprietary codes includes applying the vector embedding function to textual descriptions or variables of each of the unmapped proprietary codes. The system may generate a vector embedding for a particular unmapped proprietary code at least by applying the vector embedding function to an aggregate of the text of the particular unmapped proprietary code. Alternatively, or in addition, the system may apply the vector embedding function to each instance of the particular unmapped proprietary code and combine the resulting vector embeddings to generate the vector embedding for the particular unmapped proprietary code. The text associated with each unmapped proprietary code may be pre-processed or otherwise normalized prior to application of the vector embedding function. Pre-processing or normalizing may include, for example, filtering out certain words, handling special characters, and replacing abbreviations with full form text.

In an embodiment, the vector embedding function includes a machine learning model. The system trains the machine learning model based on a training dataset that includes mapped proprietary codes and corresponding vector embeddings. The system may receive feedback on the accuracy of results generated by applying the trained machine learning model to a set of unmapped proprietary codes. The system retrains the machine learning model based on the feedback to update the machine learning model.

In an embodiment, the system compares a target vector embedding for a target standard code to the vector embeddings computed for each of the unmapped proprietary codes. Based on a similarity measure between the target vector embedding and the vector embeddings for the unmapped proprietary codes, the system selects a subset of the unmapped proprietary codes for recommending to the user as a set of candidate unmapped proprietary codes for the target standard code. Upon receipt of user input selecting a particular unmapped proprietary code, of the set of candidate unmapped proprietary codes, the system stores an association, or mapping, between the particular unmapped proprietary code and the target standard code.

One or more embodiments described in this Specification and/or recited in the claims may not be included in this General Overview section.

### 2. Event Code Mapping System

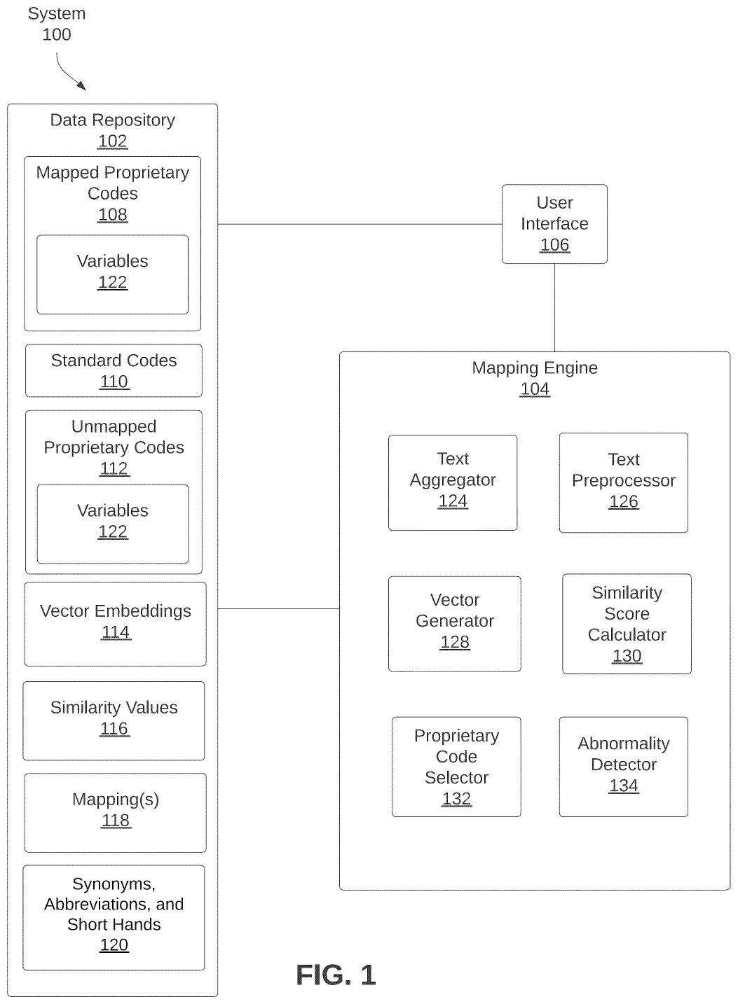

FIG. 1 illustrates a mapping system 100 in accordance with one or more embodiments. As illustrated in FIG. 1, system 100 includes a data repository 102, a mapping engine 104, and a user interface 106. In one or more embodiments, the system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, a data repository 102 is any type of storage unit and/or device (e.g., a file system, database, collection of tables, or any other storage mechanism) for storing data. Further, a data repository 102 may include multiple different storage units and/or devices. The multiple different storage units and/or devices may or may not be of the same type or located at the same physical site. Further, a data repository 102 may be implemented or executed on the same computing system as the mapping engine 104 and the user interface 106. Alternatively, or additionally, a data repository 102 may be implemented or executed on a computing system separate from the mapping engine 104 and the user interface 106. The data repository 102 may be communicatively coupled to the mapping engine 104 and the user interface 106 via a direct connection or via a network.

In embodiments, the data repository 102 is populated with information from a variety of sources and/or systems. The data repository 102 may be populated with data such as mapped proprietary codes 108, standard codes 110, unmapped proprietary codes 112, vector embeddings 114, similarity values 116, mappings 118, and synonyms, abbreviations, and short hands 120. The information may be structured (e.g. a table).

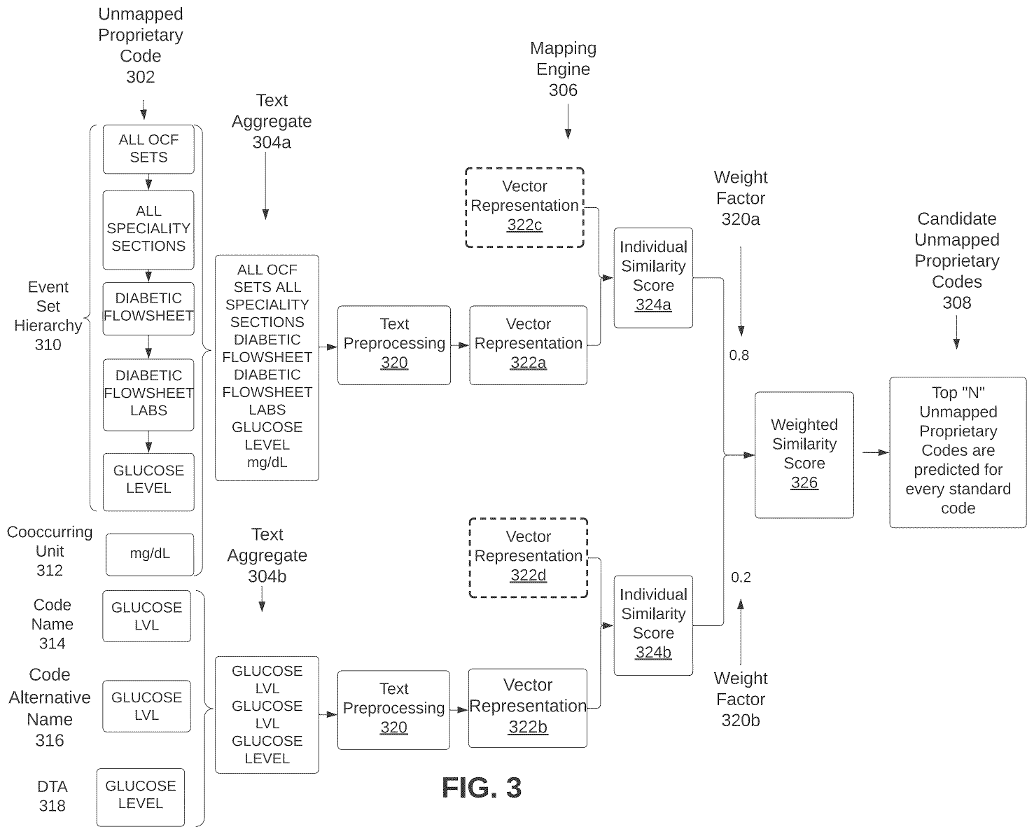

In some embodiments, the mapped proprietary codes 108 include variables 122 or attributes, i.e., reference data, for clinical and/or non-clinical events. The mapped proprietary codes 108 may be sourced from one or more disparate consumer databases. In some embodiments, the mapped proprietary codes 108 are proprietary codes that are mapped to one or more standard codes 110. The variables 122 for each of the mapped proprietary codes 108 may include an event set hierarchy and/or reference data. The event set hierarchies are a hierarchical or parent/child relationship of events sets. For example, an event set hierarchy shown in FIG. 3 includes, from general to most specific, ALL OCF (Object, Class, Function) SETS; ALL SPECIALTY SECTIONS; DIABETIC FLOWSHEET; DIABETIC FLOWSHEET LABS; and GLUCOSE LEVEL. The reference data may be consumer specific codes, industry codes, and/or unit measurement types. In embodiments, the reference data include the following fields, CODE_NAME; CODE_ALTERNATE_NAME; CONCEPT_NAME, DTA_CODE_NAME; and CO-OCCURRING UNIT CODE. Because the mapped proprietary codes 108 may be received from disparate sources, not every field of a clinical or non-clinical event will include an entry.

In embodiments, the standard codes 110 are a set of codes in which each standard code is directed to a single concept. Concepts that might not otherwise have an industry standardized code are each provided with a standard code. An example of standard codes is the Concept Cerner Knowledge Index (CCKI). The CCKI includes standard codes for concepts not having an industry standard, e.g., abnormal bleeding, birth complications, 6-minute walk start time. Each standard code 110 is mapped to one or more mapped proprietary codes 108.

In some embodiments, the unmapped proprietary codes 112 include variables 122 or attributes, i.e., reference data, for clinical and non-clinical events. The unmapped proprietary codes 112 are a set of codes developed by a specific healthcare organization or vendor to describe clinical events or other medical information in a standardized way within their own electronic health record system or clinical documentation software. The unmapped proprietary codes 112 are generally designed to be more specific and relevant to the particular healthcare organization's needs. An example of a set of proprietary codes is Code Set 72. The variables 122 for each of the unmapped proprietary codes 112 may include an event set hierarchy and/or reference data. As will become apparent from the below description, when an unmapped proprietary code is mapped to a standard code, the unmapped proprietary code becomes a mapped proprietary code.

In some embodiments, the vector embeddings 114 are text that have been converted to a numeric format. The vector embeddings 114 are representations of individual words for text analysis, typically in the form of a real-valued vector. The vector embeddings 114 may represent individual text or may represent an aggregation of text. As will be described in further detail below with respect to mapping engine 104, the vector embeddings 114 may be formed using various word embedding techniques. The vector embeddings 114 represent the standard codes 110 and the unmapped proprietary codes 112.

In embodiments, the similarity values 116 provide an indication of the similarity between the vector embeddings 114 of a standard code and an unmapped proprietary code 112. The higher the similarity values 116, i.e., the closer to 1.0, the greater a semantic match between the vector embeddings 114. The similarity values 116 may each be assigned a ranking category. For example, a similarity value less than 0.90 may be categorized as “low”, a similarity value equal to or greater than 0.90 and less than 0.98 may be categorized as “medium”, and a similarity value greater than or equal to 0.98 may be categorized as “high”. The similarity values 116 may be weighted to reflect the relevance of the type of data used to calculate the vector embeddings. For example, data with a high relevance to determining an appropriate mapping of a standard code may receive a weight of 0.8, while data with less relevance to the mapping may receive a weight of 0.2.

In embodiments, mappings 118 include mappings between the mapped proprietary codes 108 and the standard codes 110. In some embodiments, mappings 118 include mappings between standard codes 110 and the previous unmapped proprietary codes 112. As noted above, when an unmapped proprietary code is mapped to a standard code, the unmapped proprietary code becomes a mapped proprietary code.

In some embodiments, the synonyms, abbreviations, and short hands 120 are included in a table that provides synonyms, abbreviations, and/or short hands, which may or may not be specific to a consumer, and corresponding expansions for the respective synonym, abbreviation, or shorthand. For example, “SBP” may correspond to “systolic blood pressure”; “LMP” may correspond to “last menstrual period”; “I:E” may correspond to “inspiratory to expiratory ratio”; and “GAD7” may correspond to “general anxiety disorder”.

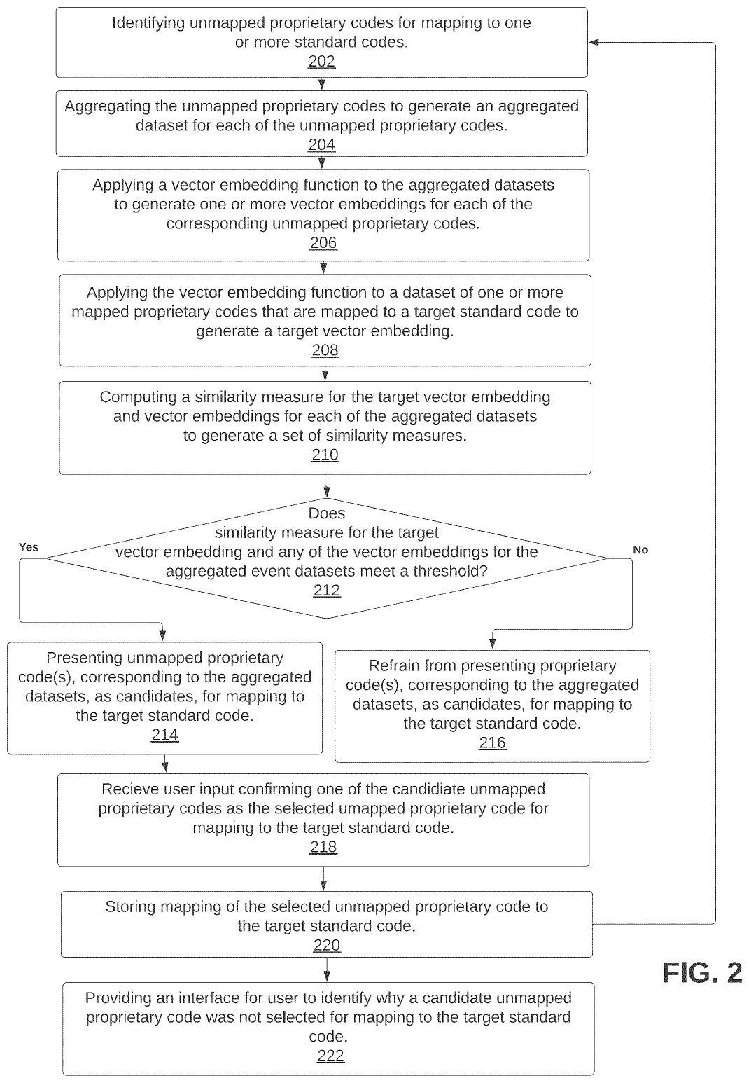

In embodiments, the mapping engine 104 is hardware and/or software configured to map standard codes 110 to unmapped proprietary codes 112. Examples of operations for providing recommendations of candidate unmapped proprietary codes are described below with reference to FIG. 2. The mapping engine 104 may include a text aggregator 124, a text preprocessor 126, a vector generator 128, a similarity score calculator 130, a proprietary code selector 132, and an abnormality detector 134. The text aggregator 124 aggregates text from the variables 122, i.e., the event set hierarchies and reference data, of the mapped proprietary codes 108 mapped to a standard code 110. The text aggregator 124 may aggregate text prior to preprocessing the text by the text preprocessor 126, after preprocessing the text by the text preprocessor 126, or before and after the text preprocessing.

In some embodiments, prior to applying the vector generator 128 to text to generate vector embeddings 114, the text is processed by the text preprocessor 126. The text preprocessor may perform functions such as converting the text into lower case and/or retaining numeric tokens. Text is converted to lower case to provide uniformity to the text. In prior art mapping engines, numeric tokens are typically removed during text preprocessing. However, removal of numeric tokens may eliminate a distinguishing feature of a concept. For example, “Right Ear 500 Hz POC” and “Right Ear 1000 Hz POC” are only differentiated using a numeric token. Accordingly, retaining numeric tokens avoids misclassifications.

In embodiments, text preprocessing may further include handling special characters, removing unwanted text from the event set hierarchy, and custom preprocessing. Handling special characters includes addressing symbols and special characters. For example, text line “D-Dimer” requires special attention. Simply replacing the “-” with a blank space creates two different tokens, namely, “D” and “Dimer”. As such, using traditional text preprocessing, the entire context of “D-Dimer” is lost. By addressing special characters, the context of the terms is maintained. Removing unwanted text from the event set hierarchy includes removing text that is present in all event set hierarchy data. Specifically, there are core event sets that are present in all event set hierarchy data. Since the core event sets do not add any new information between datasets, the core event sets are removed from the data. Custom preprocessing includes attending to consumer specific text such as synonyms, abbreviations, and short hands. The custom preprocessing may consult the synonyms, abbreviations, and short hands 120 stored in the data repository 102 to provide expansions for various consumer specific synonyms, abbreviations, and short hands.

In some embodiments, the vector generator 128 includes software and/or hardware for performing one or more vector embedding functions. Vector embedding functions are mathematical functions that map objects, such as words, sentences, or other data points, into vector representations in a multi-dimensional space. These vector representations are used to capture the semantic or contextual meaning of the objects in a numerical format that can be easily processed by machine learning algorithms.

In some embodiments, the vector embedding functions are word embedding techniques. Word embedding techniques use natural language processing (NLP) and machine learning to represent words as dense vectors of real numbers. Word embedding techniques aim to capture the semantic and syntactic meaning of words, as well as their relationships with other words in a language. Word embedding techniques include Term Frequency-Inverse Document Frequency (TF-IDF), Word2Vec, Global Vectors (GLOVE), BioWordVec fastText, and Self-Alignment Pretraining for Biomedical Entity Representations (SAPBERT).

Each of these word embedding techniques includes salient features. The TF-IDF model is designed to give more weight to the words which are very specific to certain documents whereas to give less weight to the words which are more general and occur across most documents. The Word2Vec model represents words in the form of dense vectors by capturing syntactic (grammar) and semantic (meaning) relationships. Given a large enough dataset, the Word2vec model provides strong estimates about a word's meaning based on its occurrences in the text. The GLOVE model is an unsupervised learning model which can be used to obtain dense word vectors like the Word2 Vec model. The GLOVE model first creates a huge word-context co-occurrence matrix consisting of pairs (word, context). Each element in this matrix represents how often a word occurs within the context (which can be a sequence of words) and then applies matrix factorization to approximate this matrix. The BioWordVec fastText model is 200-dimensional word embeddings trained on PubMed and MIMIC-III data and is the extension of the original BioWordVec which provides fastText word embeddings trained using PubMed and MeSH. A subword embedding model used by the BioWordVec fastText model better handles Out of Vocabulary (OOV) tokens and improves the quality of the word embeddings. The SAPBERT is a pre-trained BERT model that self-aligns the representation space of biomedical entities. The SAPBERT model leverages UMLS, a massive collection of biomedical ontologies with 4M+ concepts. The SAPBERT model can accurately capture fine-grained semantic relationships, and heterogeneous naming in the biomedical domain compared to other variants of BERT like BIO-BERT, Clinical-BERT, etc.).

In embodiments, the similarity score calculator 130 calculates a cosine similarity between vector embeddings for standard codes and vector embeddings for unmapped proprietary codes. The similarity values 116 calculated by the similarity score calculator may include cosine similarities. Cosine similarities are defined as the cosine of the angle between vectors. The cosine similarity is described mathematically as the division between the dot product of vectors and the product of the Euclidean norms or magnitude of each vector.

$$similarity ( x , y ) = cos ( θ ) = \frac{x \cdot y}{\vert x \vert \vert y \vert}$$

The similarity score calculator may weigh the cosines similarities to reflect relevance of the data used to calculate the vector embeddings.

In some embodiments, recommendations for unmapped proprietary codes are provided by the proprietary code selector 132. The proprietary code selector 132 presents candidate unmapped proprietary codes to the user interface 106 based on the similarity values 116 provided by the similarity score calculator 130. The proprietary code selector 132 may present an “N” number of candidate unmapped proprietary codes ranked by the similarity values between the vector embedding of the candidate unmapped proprietary code and the vector embedding of the standard code. Alternatively, the proprietary code selector 132 may present all candidate unmapped proprietary codes having a similarity measure with the standard code above a threshold measure.

In some embodiments, the proprietary code selector 132 provides recommendations of one or more candidate standard codes for each unmapped proprietary code 112. The candidate standard codes may be presented in any or all of the same manners as described above with the candidate unmapped proprietary codes.

In some embodiments, abnormalities in the recommendations may be identified by the abnormality detector 134. The abnormality detector 134 scans the data to identify instances where a correct definition was not followed, or an incorrect mapping was presented. Examples of abnormalities include, Systolic Blood Pressure being mapped to Diastolic Blood Pressure, Pressure Support+PEEP mapped to Pressure Support-PEEP, Activated Clotting Time mapped to Asthma Control Test, multiple standard codes for one concept, and an unmapped proprietary code mapped to multiple standard codes. The abnormality detector 134 may include features for a user to rectify the abnormalities and/or provide a justification for why the abnormality occurred. The mapping engine 104 may be modified to account for the detected abnormalities.

In an embodiment, the mapping engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, user interface 106 refers to hardware and/or software configured to facilitate communications between a user and mapping engine 104. User interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of user interface 106 are specified in different languages. The behavior of user interface elements is specified in a dynamic programming language, such as JavaScript. The content of user interface elements is specified in a markup language, such as hypertext markup language (HTML) or XML User Interface Language (XUL). The layout of user interface elements is specified in a style sheet language, such as Cascading Style Sheets (CSS). Alternatively, user interface 106 is specified in one or more other languages, such as Java, C, or C++.

### 3. Recommending Candidate Unmapped Proprietary Codes for Mapping to Standard Codes

FIG. 2 illustrates an example set of operations for recommending candidate unmapped proprietary codes for mapping to standard codes in accordance with one or more embodiments. One or more operations illustrated in FIG. 2 may be modified, rearranged, or omitted all together. Accordingly, the particular sequence of operations illustrated in FIG. 2 should not be construed as limiting the scope of one or more embodiments.

In some embodiments, the mapping engine queries the data repository to identify unmapped proprietary codes (Operation 202). Each unmapped proprietary code awaiting mapping to a standard code. As described above, a standard code represents a clinical concept. Each of the unmapped proprietary codes includes a dataset including an event set hierarchy and a dataset comprising reference data. Not all event set hierarchies will have the same depth and the reference data may not include an entry in each field.

In some embodiments, the text aggregator of the mapping engine aggregates the dataset of the same unmapped proprietary codes to generate an aggregated dataset for each of the unmapped proprietary codes (Operation 204). The aggregated dataset may include an aggregation of text from both the datasets of the event set hierarchies and the datasets comprising the reference data. Alternatively, the aggregated datasets may include a first aggregation of text from the datasets of the event set hierarchies and a second aggregation of text from the dataset of the reference data.

In some embodiments, the mapping engine applies a vector embedding function, i.e., a word embedding technique, to the aggregated text from the datasets representing unmapped proprietary codes (Operation 206). The vector embedding function generates a vector embedding for each of the unmapped proprietary codes. The vector embeddings are numerical representations of the aggregated text. Word embedding techniques include Term Frequency-Inverse Document Frequency (TF-IDF), Word2Vec, Global Vectors (GLOVE), BioWordVec fastText, and Self-Alignment Pretraining for Biomedical Entity Representations (SAPBERT).

In some embodiments, the mapping engine applies the same vector embedding function that was applied to the datasets of the unmapped proprietary codes to a dataset of one or more mapped proprietary codes that are mapped to a target standard code (Operation 208). The vector embedding function generates a target vector embedding for the target standard code. The dataset of the one or more mapped proprietary codes that are mapped to the target standard code may include aggregated text from an event set hierarchy and/or reference data of the one or more mapped proprietary codes that are mapped to the target standard code.

In some embodiments, the similarity calculator of the mapping engine computes a similarity measure for the target vector embedding and the vector embeddings for the aggregated event datasets and generates a set of similarity values (Operation 210). The similarity values are a semantic similarity between the target vector embedding and the vector embeddings for each of the unmapped proprietary codes.

In some embodiments, the mapping engine determines if the similarity measure for the target vector embedding and any of the vector embeddings for the unmapped proprietary codes meet a threshold (Operation 212). If any of the similarity values meet the threshold, the unmapped proprietary codes corresponding to the aggregated dataset are presented as candidates for mapping to the target standard code (Operation 214). If any of the similarity values do not meet the threshold, the unmapped proprietary codes corresponding to the aggregated set are not presented as candidates for mapping to the target standard code (Operation 216). The candidate unmapped proprietary codes may be presented on a user interface.

In some embodiments, subsequent to receiving the candidate unmapped proprietary codes, a user may provide input confirming one of the candidate unmapped proprietary codes as the selected unmapped proprietary code for mapping to the target standard code (Operation 218). The user input may include selecting an icon representing the desired candidate unmapped proprietary code.

The mapping of the selected unmapped proprietary code to the target standard code may be stored in the data repository (Operation 220). In this manner, the mapping of the unmapped proprietary code to the target standard code may be used in subsequent applications of the vector embedding function. More particularly, the selected unmapped proprietary code becomes a mapped proprietary code that may be used, as described above, in generating a target vector. The mapping of the target standard code to the selected unmapped proprietary code provides an additional dataset for processing of the previously unmapped proprietary code and mapped standard code for use in future mappings. The additional dataset for the previously unmapped proprietary code and the standard code increases the accuracy and precision of future recommendations.

In some embodiments, to better understand why the user did not select a particular candidate unmapped proprietary code, the user is prompted to identify why a candidate unmapped proprietary code was not selected (Operation 222). The user prompt may include an assortment of predefined user selectable responses and/or an input box for text entry.

In some embodiments, prior to the vector embedding function being applied to the aggregated dataset, a token is identified for each word/text of the aggregated dataset. The vector embedding function is then applied to each of the tokens to generate a set of vector embeddings. A representative vector embedding may then be generated from the set of vector embeddings. Alternatively, the text of the aggregated dataset may be treated as a single token, with the vector embedding function being applied to the single token.

In some embodiments, the vector embedding function includes a machine learning model. The machine learning models are trained on training datasets to compute vector embeddings from mapped proprietary codes. Particular training data, of the training datasets, may include one or more historical mapped proprietary codes, and a vector embedding corresponding to the historical mapped proprietary codes. Applying the vector embedding function to the dataset of the first unmapped proprietary code includes applying the machine learning model to the dataset of the first unmapped proprietary code, receiving feedback based on an accuracy of results generated by applying the vector embedding function, and retraining the machine learning model based on the feedback.

In some embodiments, the similarity values are cosine similarity values for the target vector embedding and the vector embeddings for the candidate unmapped proprietary codes. The cosine similarity values may be calculated for the vector embedding representing the aggregated dataset of the target standard code and the aggregated dataset of each of the candidate unmapped proprietary codes. Alternatively, the cosine similarity values may be weighted based on relevance of the datasets. For example, the dataset for each of the target standard code and each of the candidate unmapped proprietary codes taken from the event set hierarchy of the respective target standard code and the respective reference data may have a low/lower relevancy in precisely and accurately determining a candidate unmapped proprietary code. Conversely, the dataset for each of the target standard code and each of the candidate unmapped proprietary codes taken from the referenced data of the respective standard and unmapped proprietary codes may have a high/higher relevancy in precisely and accurately determining candidate unmapped proprietary codes.

In some embodiments, prior to applying the vector embedding function to the dataset of the target standard code and the datasets of the candidate unmapped proprietary codes, the text of the dataset is processed. Preprocessing of the text may include converting text data into lowercase letters to provide uniformity in the text, and retaining numeric tokens that, in prior mapping engines, would have been removed. As described above, the numeric tokens provide context to the text.

Text preprocessing may further include handling special characters such as removing unwanted text from the event set hierarchy. Additionally, preprocessing the text may include custom reprocessing for synonyms, abbreviations, and short hands. The synonyms, abbreviations, and short hands may be consumer specific and/or industry standards. The synonyms, abbreviations, and short hands may be provided in a table format along with their respective expansions.

In some embodiments, the operations for recommending candidate unmapped proprietary codes further includes identifying “N” highest similarity values of the similarity values, and presenting unmapped proprietary codes, mapped to vector embeddings that correspond to the “N” highest similarity values, as candidate unmapped proprietary codes for mapping to the target standard code.

### 4. Example Embodiment

A detailed example is described below for purposes of clarity. Components and/or operations described below should be understood as one specific example which may not be applicable to certain embodiments. Accordingly, components and/or operations described below should not be construed as limiting the scope of any of the claims.

FIG. 3 illustrates processing of clinical data representing unmapped proprietary codes to provide recommendations of candidate unmapped proprietary codes 308. Initially, clinical data or variables for an unmapped proprietary code 302 is targeted. The variables include an event set hierarchy 310, cooccurring unit 312, code name 314, code alternative name 316, and Discrete Task Assay (DTA) 318. Although shown including an entry in each of the data fields, it is understood that not all data fields may include an entry.

The text from each of the event set hierarchy 310 and the cooccurring unit 312 are combined into a first text aggregate 304a and the text from each of the code name 314, code alternative name 316, and Discrete Task Assay (DTA) 318 are combined into a second aggregate text 304b.

Each of the first and second aggregated texts 304a, 304b are then fed into the mapping engine 306. Text preprocessing 320 occurs on the text of the first and second aggregated texts 304a, 304b to provide uniformity and to address special characters, synonyms, abbreviations, short hands.

Upon completion of the text preprocessing 320, the mapping engine 306 generates vector representations 322a, 322b of the respective first and second aggregated texts 304a, 304b. The vector representations 322a, 322b may be of individual tokens within the respective first and second aggregated texts 304a, 304b or of the entirety of the respective first and second aggregated texts 304a, 304b.

The vector representations 322a. 322b for the first and second aggregated texts 304a. 304b, respectively, are then compared to vector representations 322c, 322d, respectively, of first and second aggregated texts (not shown) of datasets of mapped propriety codes (not shown) mapped to a target standard code to determine individual similarity scores 324a of the first aggregated texts and the individual similarity scores 324b of the second aggregated texts.

Based on the suggestions of the Subject Matter Expert (SME) and Exploratory Data Analysis (EDA) it was found that Code Name, Code Alternate Name, and DTA information contain more relevant information that helps in aligning to a correct standardized event code (CCKI). Hence a weight factor 320b of “0.8” was used for Code Name, Code Alternate Names, DTA, and Concept Name, and a weight factor 320b of “0.2” was used for Cooccurring Units and ESH. The weight factors 320a, 320b are applied to the respective individual similarities scores 324a, 324b of the first and second aggregated texts 304a, 304b, respectively, to generate a first and second weighted similarity scores which are then combined to create a weighted similarity score 326. The higher the weighted similarity score 326, the higher the semantic match between the unmapped proprietary code and the target standard code.

Weighted similarity scores are then calculated for each of unmapped proprietary codes and the target standard code. The top “N” candidate unmapped proprietary codes 308 are provided for mapping to the target standard code.

This process may be repeated with each standard code to provide the top “N” candidate unmapped proprietary codes for each standard code.

### 5. Recommendation Interface

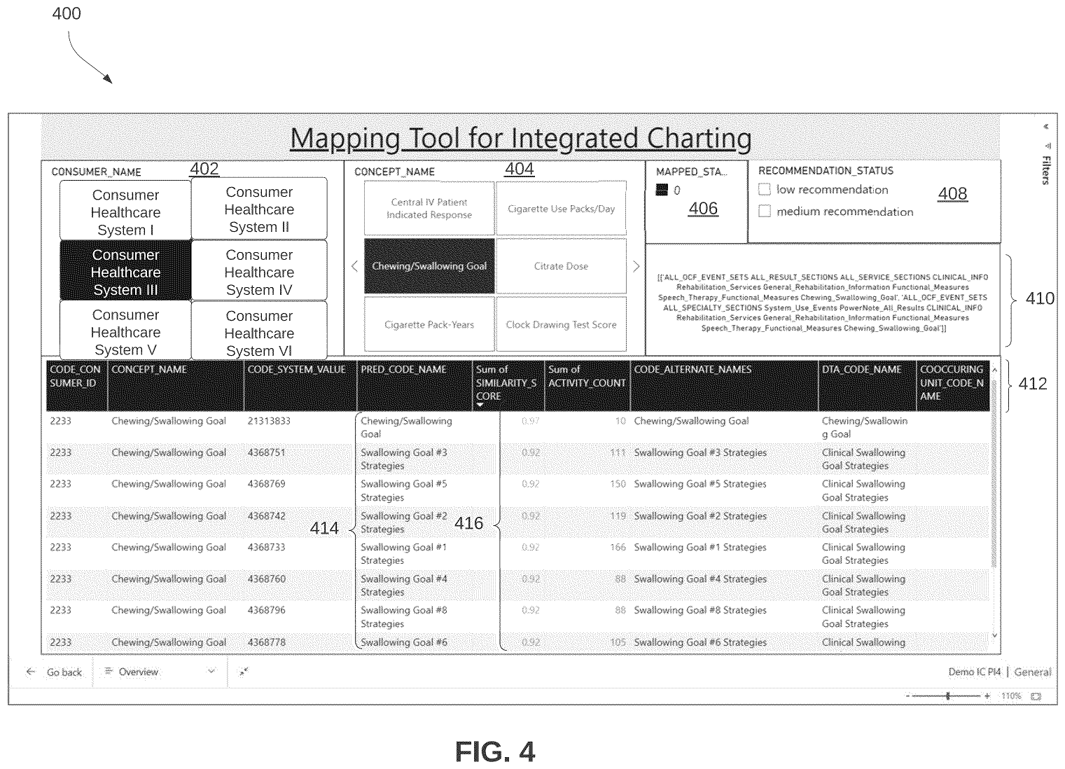

FIG. 4 illustrates an example of a recommendation interface 400 in accordance with one or more embodiments. The recommendation interface 400 may display information in a table format for easy viewing.

The interface 400 provides indication of a consumer name 402, a concept name 404, mapping status 406, a recommendation status 408, aggregated text 410, and code headings 412. The concept name 404, identified as “Chewing/Swallowing Goal”, is the target standard code. As shown, the candidate unmapped proprietary codes are presented in PRED_CODE_NAME 414. The candidate unmapped proprietary codes are presented in ranked order based on similarities scores 415. Although eight (8) candidate unmapped proprietary codes are shown, it is envisioned that more or less than eight (8) candidate unmapped proprietary codes may be presented.

### 6. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

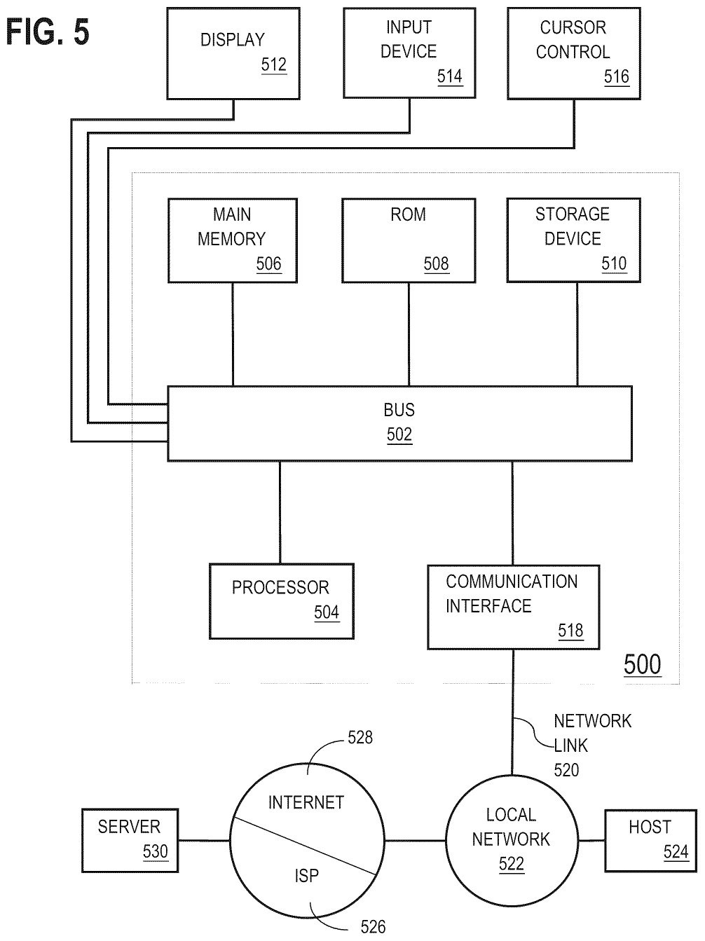

For example, FIG. 5 is a block diagram that illustrates a computer system 500 upon which an embodiment of the invention may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or solid state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic, or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the worldwide packet data communication network now commonly referred to as the “Internet” 528. Local network 522 and Internet 528 both use electrical, electromagnetic, or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

### 7. Miscellaneous; Extensions

Embodiments are directed to a system with one or more devices that include a hardware processor and that are configured to perform any of the operations described herein and/or recited in any of the claims below.

In an embodiment, a non-transitory computer readable storage medium comprises instructions which, when executed by one or more hardware processors, causes performance of any of the operations described herein and/or recited in any of the claims.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A non-transitory computer readable medium comprising instructions which, when executed by one or more hardware processors, cause performance of operations comprising:
identifying a plurality of unmapped proprietary codes for mapping to standard codes;
generating a plurality of vector embeddings corresponding to the plurality of unmapped proprietary codes, wherein generating the plurality of vector embeddings comprises:
generating a first vector embedding for a first unmapped proprietary code of the plurality of unmapped proprietary codes at least by:
applying a vector embedding function to a dataset of the first unmapped proprietary code;
generating a target vector embedding for a target standard code at least by:
applying the vector embedding function to a dataset of mapped proprietary codes mapped to the target standard code to generate a target set of one or more vector embeddings;
computing a similarity measure for the target vector embedding and each of the plurality of vector embeddings to generate a plurality of similarity values, the plurality of similarity values comprises:
a first similarity measure for the target vector embedding and the first vector embedding; and
based at least on the first similarity measure, presenting the first unmapped proprietary code as a candidate unmapped proprietary code for mapping to the target standard code.

2. The medium of claim 1, wherein generating the first vector embedding comprises:
applying the vector embedding function to the dataset of the first unmapped proprietary code to generate a first set of vector embeddings; and
generating the first vector embedding based on the first set of vector embeddings.

3. The medium of claim 1, wherein generating the target vector embedding comprises:
applying the vector embedding function to the dataset of mapped proprietary codes mapped to the target standard code to generate a target set of vector embeddings; and
generating the target vector embedding based on the first target set of vector embeddings.

4. The medium of claim 1, wherein the vector embedding function comprises a machine learning model, and wherein the operations further comprise:
training the machine learning model based on training datasets to compute vector embeddings from mapped proprietary codes, wherein particular training data, of the training datasets, comprises:
one or more historical mapped proprietary codes;
a vector embedding corresponding to the historical mapped proprietary codes;
wherein applying the vector embedding function to the dataset of the first unmapped proprietary code comprises applying the machine learning model to the dataset of the first unmapped proprietary code;
receiving feedback based on an accuracy of results generated by applying the vector embedding function; and
retraining the machine learning model based on the feedback.

5. The medium of claim 1, wherein generating the plurality of vector embeddings further comprises:
generating a second vector embedding for a second unmapped proprietary code of the plurality of unmapped proprietary codes at least by:
applying the vector embedding function to a dataset of the second unmapped proprietary code;
wherein the plurality of similarity values further comprises:
a second similarity measure for the target vector embedding and the second vector embedding;
wherein the operations further comprise:
based at least on the second similarity measure, refraining from presenting the second unmapped proprietary code as any candidate unmapped proprietary code for mapping to the target standard code.

6. The medium of claim 1, wherein applying the vector embedding function to the first unmapped proprietary code comprises:
aggregating the dataset of the first unmapped proprietary code into an aggregated data record;
identifying a plurality of tokens based on the aggregated data record; and
applying the machine learning model to each token of the plurality of tokens to generate the first set of vector embeddings.

7. The medium of claim 1, wherein applying the vector embedding function to the dataset of the first unmapped proprietary code comprises applying the vector embedding function to an aggregated set of text that is generated by aggregating text corresponding to each dataset of the first unmapped proprietary code.

8. The medium of claim 1, wherein the first similarity measure comprises a weighted cosine similarity measure for the target vector embedding and the first vector embedding.

9. The medium of claim 1, wherein the operations further comprise:
prior to applying the vector embedding function to the first unmapped proprietary code, pre-processing the dataset of the first unmapped proprietary code at least by:
(a) converting text data into lowercase,
(b) retaining numeric tokens,
(c) handling special characters,
(d) removing unwanted text from event set hierarchy, and
(e) custom reprocessing for synonyms, abbreviations, and short hands.

10. The medium of claim 1, wherein the operations further comprise:
identifying “N” highest similarity values of the plurality of similarity values; and
presenting unmapped proprietary codes, mapped to vector embeddings that correspond to the “N” highest similarity values, as candidate unmapped proprietary codes for mapping to the target standard code.

11. The medium of claim 1, wherein the operations further comprise:
identifying a subset of similarity values, of the plurality of similarity values, that meet a threshold similarity values; and
presenting unmapped proprietary codes, mapped to vector embeddings that correspond to the subset of similarity values, as candidate unmapped proprietary codes for mapping to the target standard code.

12. The medium of claim 1, wherein applying the vector embedding function to a dataset of mapped proprietary codes comprises using one or more of the following word embedding techniques: BioWordVec fastText or Self-Alignment Pretraining for Biomedical Entity Representations (SAPBERT).

13. The medium of claim 1, wherein the operations further comprise:
selecting the first unmapped proprietary code for mapping to the target standard code.

14. A method comprising:
identifying a plurality of unmapped proprietary codes for mapping to standard codes;
generating a plurality of vector embeddings corresponding to the plurality of unmapped proprietary codes, wherein generating the plurality of vector embeddings comprises:
generating a first vector embedding for a first unmapped proprietary code of the plurality of unmapped proprietary codes at least by:
applying a vector embedding function to a dataset of the first unmapped proprietary code;
generating a target vector embedding for a target standard code at least by:
applying the vector embedding function to a dataset of mapped proprietary codes mapped to the target standard code to generate a target set of one or more vector embeddings;
computing a similarity measure for the target vector embedding and each of the plurality of vector embeddings to generate a plurality of similarity values, the plurality of similarity values comprises:
a first similarity measure for the target vector embedding and the first vector embedding; and
based at least on the first similarity measure, presenting the first unmapped proprietary code as a candidate unmapped proprietary code for mapping to the target standard code,
wherein the method is performed by at least one device including a hardware processor.

15. The method of claim 14, wherein generating the first vector embedding comprises:
applying the vector embedding function to the dataset of the first unmapped proprietary code to generate a first set of vector embeddings; and
generating the first vector embedding based on the first set of vector embeddings.

16. The method of claim 14, wherein generating the target vector embedding comprises:
applying the vector embedding function to the dataset of mapped proprietary codes mapped to the target standard code to generate a target set of vector embeddings; and
generating the target vector embedding based on the first target set of vector embeddings.

17. The method of claim 14, wherein the vector embedding function comprises a machine learning model, and further comprising:
training the machine learning model based on a training datasets to compute vector embeddings from mapped proprietary codes, wherein a particular training data, of the training datasets, comprises:
one or more historical mapped proprietary codes;
a vector embedding corresponding to the historical mapped proprietary codes;
wherein applying the vector embedding function to the dataset of the first unmapped proprietary code comprises applying the machine learning model to the dataset of the first unmapped proprietary code;
receiving feedback based on an accuracy of results generated by applying the vector embedding function; and
retraining the machine learning model based on the feedback.

18. The method of claim 14, wherein generating the plurality of vector embeddings further comprises:
generating a second vector embedding for a second unmapped proprietary code of the plurality of unmapped proprietary codes at least by:
applying the vector embedding function to a dataset of the second unmapped proprietary code;
wherein the plurality of similarity values further comprises:
a second similarity measure for the target vector embedding and the second vector embedding;
wherein based at least on the second similarity measure, refraining from presenting the second unmapped proprietary code as any candidate unmapped proprietary code for mapping to the target standard code.

19. The method of claim 14, wherein applying the vector embedding function to the first unmapped proprietary code comprises:
aggregating the first unmapped proprietary code into an aggregated data record;
identifying a plurality of tokens based on the aggregated data record; and
applying the machine learning model to each token of the plurality of tokens to generate the first set of vector embeddings.

20. The method of claim 14, wherein applying the vector embedding function to the dataset of the first unmapped proprietary code comprises applying the vector embedding function to an aggregated set of text that is generated by aggregating text corresponding to each dataset of the first unmapped proprietary code.

21. The method of claim 14, wherein the first similarity measure comprises a weighted cosine similarity measure for the target vector embedding and the first vector embedding.

22. The method of claim 14, wherein the operations further comprise:
prior to applying the vector embedding function to the first unmapped proprietary code, pre-processing the dataset of the first unmapped proprietary code at least by:
(a) converting text data into lowercase,
(b) retaining numeric tokens,
(c) handling special characters,
(d) removing unwanted text from event set hierarchy, and
(e) custom reprocessing for synonyms, abbreviations, and short hands.

23. The method of claim 14, further comprising:
identifying “N” highest similarity values of the plurality of similarity values; and
presenting proprietary codes, mapped to vector embeddings that correspond to the “N” highest similarity values, as candidate proprietary codes for mapping to the target standard code.

24. The method of claim 14, further comprising:
identifying a subset of similarity values, of the plurality of similarity values, that meet a threshold similarity measure; and
presenting unmapped proprietary codes, mapped to vector embeddings that correspond to the subset of similarity values, as candidate unmapped proprietary codes for mapping to the target standard code.

25. The method of claim 14, wherein applying the vector embedding function to a dataset of mapped proprietary codes comprises using one or more of the following word embedding techniques: BioWordVec fastText or Self-Alignment Pretraining for Biomedical Entity Representations (SAPBERT).

26. The method of claim 14, further comprise:
selecting the first unmapped proprietary code for mapping to the target standard code.
